(19) United States
(12) Patent Application Publication
(10) Pub. No.: US 2021/0290125 A1
(43) Pub. Date: Sep. 23, 2021

(54) METHOD AND DEVICE FOR PREPARING AND ADMINISTERING TREATMENT AGENTS USING PRODUCTS DERIVED FROM WHOLE BLOOD

(71) Applicant: Allen J Meglin, Savannah, GA (US)

(72) Inventor: Allen J Meglin, Savannah, GA (US)

(21) Appl. No.: 15/929,810

(22) Filed: May 22, 2020

Related U.S. Application Data
(60) Provisional application No. 62/904,184, filed on Sep. 23, 2019.

(51) Int. Cl. A61B 5/15; A61B 10/02

(52) U.S. Cl. CPC A61B 5/150236; A61B 5/150351; A61B 10/0283; A61B 5/150755; A61B 5/150992; A61B 5/150603

(57) ABSTRACT

The present invention relates to a device including a syringe designed to extract blood from a patient, process the blood to create a derivative blood product, and then subsequently to administer the derivative blood product. The present invention further relates to an associated method, carried out by: drawing blood, processing the drawn blood to produce a derivative blood product, and administering the derivative blood product, all using a single sterile vessel.

### BACKGROUND OF THE INVENTION

The invention relates generally to methods and apparatus for obtaining and administering derivative blood products. The invention further relates to methods and apparatus for obtaining and administering platelet-rich plasma. According to some aspects, the invention relates to methods and apparatus for obtaining and administering derivative blood products enhanced with additional agents. According to some aspects, the invention relates to such methods and apparatus for use in human subjects. According to some aspects, the invention relates to such methods and apparatus suitable for use in one or more of a clinical setting, a research laboratory setting, or any other setting in which derivative blood products are used. According to some aspects, the invention relates to such methods and apparatus suitable for diagnosing or treating a presumed or confirmed diagnosis of a condition or disease by transporting a therapeutic agent to a treatment site using derivative blood products.

### SUMMARY OF THE INVENTION

The present invention relates to a device and a method associated with the device.

With respect to the device, it is a syringe designed as both a vessel in which to create a derivative blood product, and then subsequently to be used as an administration vessel for the derivative blood product. This device can first be used to draw blood from a patient into a closed, sterile vessel. The same device can then be used as a processing vessel in a centrifuge, or any other reactor or device, for producing a derivative blood product. The same device can be used before or after processing to draw other treatment agents into the blood drawn from the patient by simply changing needles on the syringe without transferring the contents of the closed sterile vessel in or out during the process. The same device can re-inject the derivative blood product from the vessel in which the blood was collected and processed directly into the same patient. This eliminates the need for up to three separate vessels/devices, namely a collection vessel, a separate processing vessel, and a third derivative blood product administration vessel.

The core components of one embodiment of the invention are a dual-Luer lock syringe, with a male access port on one side and a female access port on the other side as well as a detachable syringe plunger rod and a shuttlecock with a screw thread and rubber stopper inside. Also provided are Luer caps to seal the male and female Luer ports. Such an embodiment may be provided in kit form or as separate components obtained a la carte. The kit may also include a sterile stand, tray, or the like, for supporting the plunger rod when it is detached from the shuttlecock.

According to some embodiments and variations, the elements are configured as follows: A male Luer lock port at one end of the syringe can be attached to a standard hypodermic syringe needle for withdrawing blood. A larger bore, female Luer lock port is located at the other end of the syringe. The female opening has a diameter that allows the passage of a threaded plunger rod that can be attached to the threaded shuttlecock and rubber stopper. Either the plunger rod or the shuttlecock may have a male thread, with the other of the two components having a mating female thread. When the plunger rod is unscrewed from the shuttlecock, after withdrawing blood from the patient, the female Luer lock will accept a threaded screw cap. When the syringe needle is removed from the male Luer lock, after the blood withdrawal, the male Luer lock will also accept a threaded screw cap. The entire blood collection is held in a sterile chamber. With a cap attached at each end, it is a closed system that can be fit into a centrifuge. The male Luer lock has the usual Luer lock threads to accept flanges for of a standard syringe needle or other standardized Luer attachment, but that end of the syringe also has an extended external flange thus preventing improper placement of the syringe in the centrifuge by forcing the needle end of the capped syringe to face upward in the centrifuge. Lighter material thus collects at the end with the male Luer lock. The syringe is thus designed to leave the heavy, packed red blood cells (RBCs) at the plunger end and the lighter, platelet rich plasma (PRP) at the needle end after centrifugation. With respect to the device it should be further noted that once the blood has been spun in the centrifuge, the plunger rod can be reattached, an injection needle or other administration attachment attached to the male Luer lock, and the same syringe can then be used to administer the lighter, floating PRP. The PRP may itself be a therapeutically useful agent, or it may serve as a scaffold for an adjutant agent having therapeutic value. Before administering the PRP to the patient, such an adjutant having therapeutic value can be also brought into the now-separated PRP.

In some embodiments, the plunger rod connection with the shuttlecock or stopper may have threads arranged so the plunger rod may be pushed through the stopper so as to be out of the way of a cap over the plunger rod port for centrifugation. In other embodiments, the flanges on the outside of the syringe may be on a sliding sleeve that allows the flanges to be positioned near the plunger port of near the needle port as desired. In some embodiments, the shuttlecock may be omitted if the stopper is made of a material in which durable, reliable threads or other connection features may be formed to mate with corresponding features on the plunger rod.

With respect to the associated method, in order to carry out the method the following core steps are followed: drawing blood, centrifuging blood, and re-administering partitioned blood, all using a single vessel. The blood is drawn from patient using the syringe in a normal fashion. The flanges are preferably positioned at or positionable to the opposite end of the usual and customary syringe, that is, at the needle end, rather than forming a grip at the plunger end. Once the plunger rod is used to withdraw blood from the patient, the rod is unscrewed and removed from the syringe. The needle is also unscrewed from its Luer lock. The syringe is then capped on both sides and placed into centrifuge. The flanges positioned at the needle end keep the syringe positioned correctly in the centrifuge. If sliding flanges are used, they are first positioned to the needle end of the syringe. If detachable, the plunger rod is placed in a sterile holder. Otherwise the plunger rod is detached from the stopper and pushed into the syringe without moving the stopper so a cap may be placed over the plunger rod port. Once the syringe has been spun in the centrifuge, the plunger rod is reinserted into the syringe or withdrawn and reattached to the stopper, and used to push the spun blood product back into the patient using a sterile needle.

Ultimately, at the conclusion of these steps blood drawn from the patient can be collected and centrifuged and then re-injected back into the patient using a single sterile vessel.

It is desirable to have a device that will perform all three steps: drawing blood, centrifuging blood, and re-administering partitioned blood, all using a single vessel. Current devices require multiple vessels to first blood draw in a syringe (the first vessel), then transfer the blood into a second vessel for centrifugation, and finally draw off the desired fluid in a third vessel for re-administration. In some current methods, instead of a syringe and separate centrifugation vessel, a sample may be directly collected in a Vacutainer® (product of Becton, Dickinson and Company; available as of the application date) and centrifuged in the same vessel, but a second vessel for re-administration is still required. Furthermore, it would also be desirable to have a device that maintains sterility inside the vessel while allowing the outside of the vessel to be placed into a non-sterile centrifuge. Still further, it would be desirable to have a device that maintained the separation of the red blood cells from the platelet rich plasma and does not allow admixture of the already separated fluid and cells. This can be done with a mechanical separator or by means of a gel separator in the syringe. This can also be accomplished by creating two chambers in the syringe for physical separation. It is further advantageous to have the administration flanges at the male end (re-injection end) of the device to prevent inadvertent upside down placement of the syringe in the centrifuge. The red blood cells in the pellet should be packed against the plunger to allow administration of PRP only. The disclosed device and associated method advantageously fills these needs and addresses the aforementioned deficiencies by providing a single system for collection, centrifugation and administration of separated blood products.

In the following description reference is made to the accompanying drawings, which form a part hereof, and in which are shown example implementations. It should be understood that other implementations are possible, and that these example implementations are intended to be merely illustrative.

### BRIEF DESCRIPTION OF THE DRAWINGS

FIG. 1 is a cutaway view through a longitudinal axis of symmetry of an embodiment showing some features of the invention;

FIG. 2 is a detail showing the attachment of the plunger rod of the embodiment of FIG. 1;

FIG. 3 is a detail showing the use of Luer lock end caps to create a closed vessel using the syringe body of some embodiments;

FIG. 4 is a general view of the closed vessel being inserted into a centrifuge for centrifugation of the contents thereof;

FIG. 5 is a view an embodiment showing other features of the invention;

FIG. 6 is a view of the embodiment of FIG. 5 in which the plunger rod has been advanced into the syringe body to make room for applying a cap to the plunger rod end;

FIG. 7 shows some alternative applicators for use with the syringe in place of a hypodermic needle;

FIG. 8 is a side view showing a sliding flange to more conveniently provide both an alignment feature for centrifugation and a grip for administration of material from the syringe into a patient;

FIG. 9 is a cutaway along a central plane of symmetry of one embodiment for attaching the plunger rod to the stopper;

FIG. 10 is a cutaway along a central plane of symmetry of a second embodiment for attaching the plunger rod to the stopper;

FIG. 11 is a cutaway along a central plane of symmetry of a third embodiment for attaching the plunger rod to the stopper;

FIG. 12 is a cutaway of a conventional sample tube containing centrifuged blood;

FIG. 13 is a perspective view showing a conventional centrifuge rotor;

FIG. 14 is a cutaway of one embodiment of a syringe according to aspects of the invention containing centrifuged blood; and

FIG. 15 is a cutaway one embodiment of a syringe according to aspects of the invention containing centrifuged blood.

### DETAILED DESCRIPTION

The following section describes in detail several exemplary embodiments of the invention illustrating by example the principles of construction and usage of apparatus according to the invention, and the acts and steps of methods according to the invention. Features of the various exemplary embodiments may be further recombined to produce other exemplary embodiments of the invention, as will be evident to the skilled artisan upon reading the details thereof. The exemplary embodiments are now described with reference to the drawings enumerated above.

### Device Structures

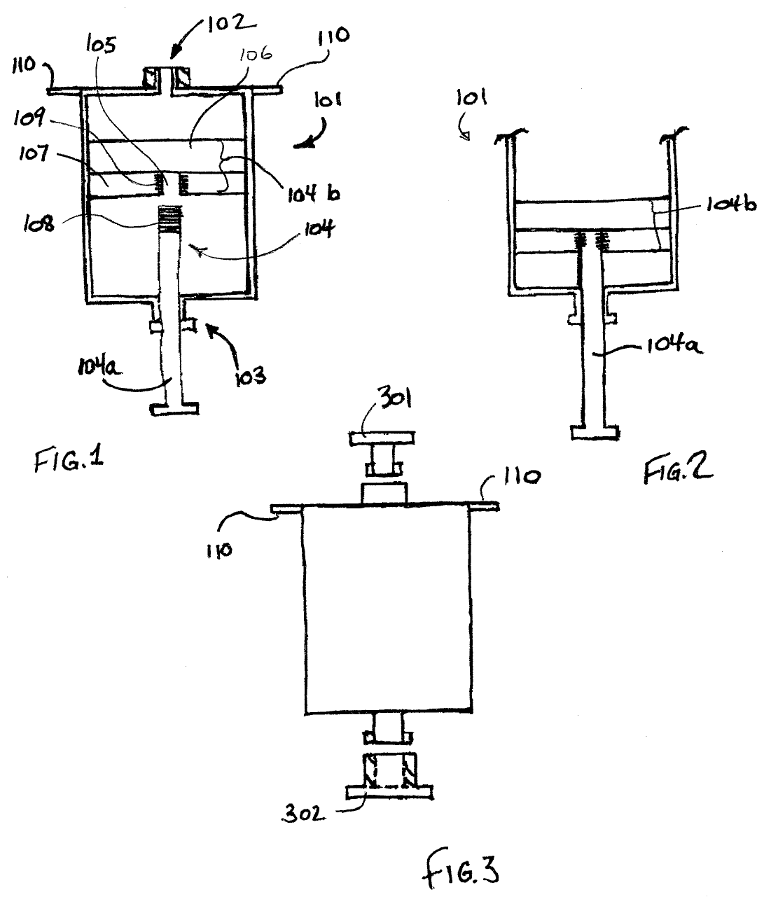

One embodiment, now described in connection with FIGS. 1-4, is a syringe 101 including an inflow port 102 adapted to receive at different times a hypodermic needle (not shown) and a closing cap 301, the syringe 101 further including a plunger port 103 and a plunger 104. The plunger port 103 is adapted to pass a plunger rod 104a into the body of the syringe 101 to removably attach to an elastomeric sliding stopper 104b within the body of the syringe. The plunger port 103 is further adapted to receive a closing cap 302 when the plunger rod 104a is not attached to the elastomeric stopper 104b. When both the inflow port closing cap 301 and the plunger port closing cap 302 are attached, the system is closed. Such a system, manufactured under sterile conditions, or sterilized after manufacture, can be kept sterile throughout its usage cycle, as will be explained below.

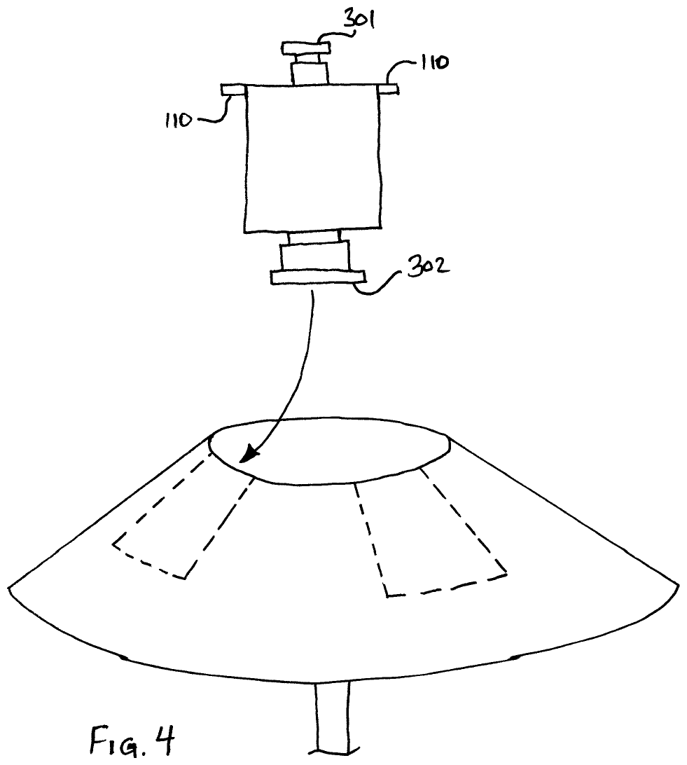

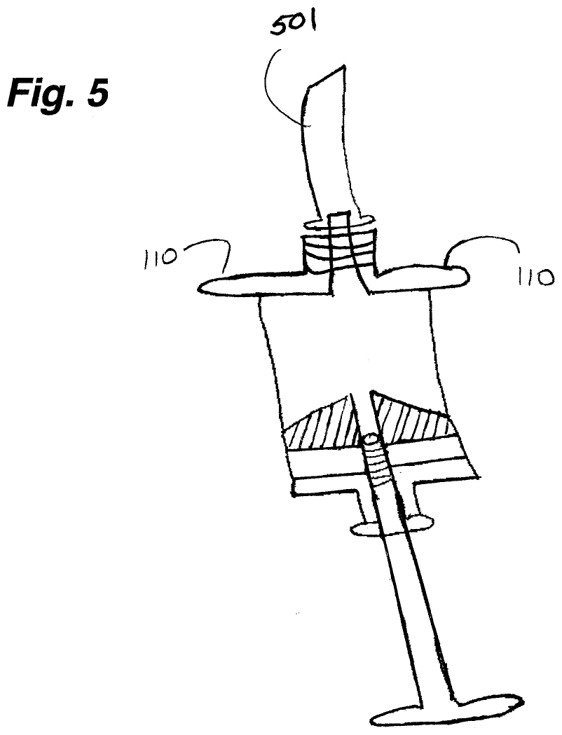

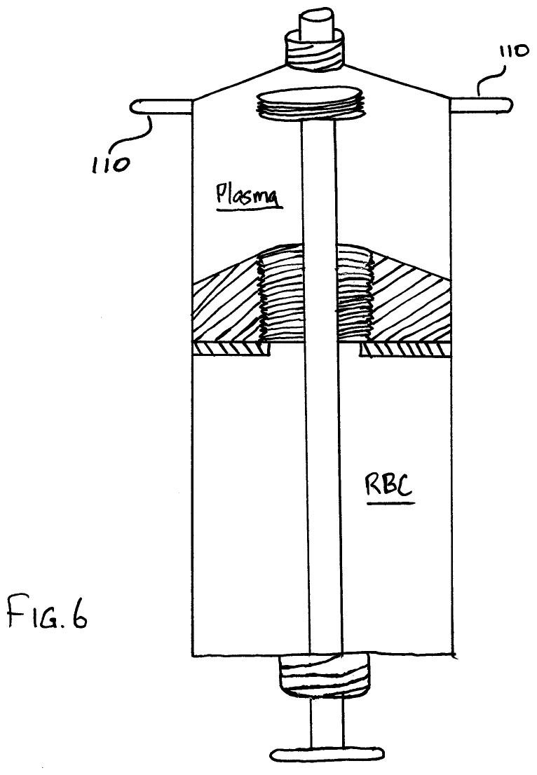

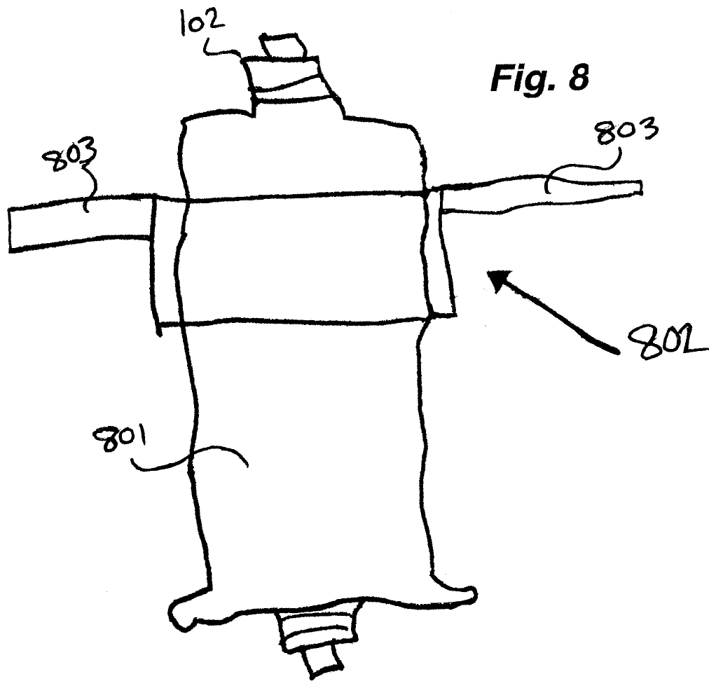

In a more complete form, the device may include one or more of the following components:
Rubber stopper with density designed to separate the RBC pellet from the PRP supernatant;
A detachable plunger rod (FIG. 1);
A threaded plunger rod whose threaded end is wider than a body portion of the rod, such that when unscrewed the threaded end is beyond the stopper, so the rod can be pushed through the stopper into the syringe for spinning (FIGS. 5 and 6);
A rubber stopper with or without a hole to allow RBCs to pass through the stopper; and
A shuttle attached to plunger to allow plunger rod threads to screw either into the stopper at the bottom of the stopper or at the top of the stopper (in embodiments in which a non-detachable plunger rod is used). (Cf. FIGS. 1 and 5; cf. FIGS. 2 and 6.)
The grip flanges of the syringe may be attached to the syringe body via a sliding sleeve that permits the flanges to be positioned at either extreme end of the syringe body. See FIG. 8.

### Plungers

Several options are available for available for attaching the plunger rod 104a to the stopper 104b, and any suitable option may be employed. Several examples, now described, include a blind, threaded hole in a stopper mated to a threaded plunger rod end as shown in FIGS. 1 and 2; a threaded through hole in a stopper mated to a threaded plunger rod end as shown in FIG. 9; a stepped through hole in a stopper mated to an enlarged threaded plunger rod end as shown in FIG. 10; and a partially threaded through hole in a stopper mated to a thread on a plunger rod having a terminal end plate as shown in FIG. 11.

FIGS. 1 and 2 show a blind hole 105 formed in the body of the stopper 104b. The hole is formed in a plastic insert 107 embedded bonded to an elastomeric body 106. Together, the plastic insert 107 and the elastomeric body 106 comprise the stopper 104b. Plunger rod 104a includes threads 108 at a distal end thereof, disposed so as to engage corresponding threads 109 formed in blind hole 105. FIG. 1 shows the plunger rod 104a disengaged from stopper 104b; FIG. 2 shows the plunger rod 104a engaged with stopper 104b.

FIG. 9 shows an open hole 902 that passes completely through the stopper 901. Hole 902 is threaded 903 at least part way through the stopper 901. Plunger rod 904 includes corresponding threads 905 for engaging threads 903. By providing threading 903 all the way through the stopper 901, the system either allows the plunger rod 904 to be pushed through the stopper 901 when the plunger rod 904 is unsecured from the stopper 901 or alternatively allows the plunger rod 904 to be withdrawn from the system when the plunger rod 904 is unsecured from threads 903.

As described in connection with FIG. 1, the stopper may include a separate shuttle providing supportive strength attached to the stopper body by any suitable means. As alternatively described in connection with FIG. 9, the stopper may be a body formed as a unit of a single material or composite having sufficient inherent strength and support characteristics.

In an alternate embodiment shown in FIG. 10, the plunger rod 1001 includes an enlarged diameter at the distal end thereof. In this example, the enlarged distal end is threaded 1002. The stopper 1006 has a hole having a first section 1003 with a diameter sufficient to pass the central body of the plunger rod 1001, and a second section 1004 with a larger diameter and threading 1005 to engage the threading 1002 of the enlarged distal end of the plunger rod 1001. When disengaged, the plunger rod 1001 can be pushed through the stopper 1006, as shown in FIG. 10 and explained further below.

In an alternate embodiment shown in FIG. 11, the plunger rod 1101 includes an end cap 1107 having a larger diameter than the central body thereof at the distal end thereof. In this example, the distal end of the central body of the plunger rod 1101 is threaded 1102 adjacent the end cap 1107. The stopper 1106 has a hole having a first section 1103 with a diameter just sufficient to pass the central body of the plunger rod 1101, and a second section 1104 with threading 1105 to engage the threading 1102 of the plunger rod 1101. When disengaged, the plunger rod 1101 can be pushed through the stopper 1106, as shown in FIG. 11 and explained further below.

In each of the foregoing examples, a bayonet mount or other mount by which the rod is releasable from the stopper without pushing or pulling on the stopper may replace the threads.

### Stoppers

Each of the stoppers of FIGS. 9, 10, and 11 include passages or holes through which blood or blood products can pass during centrifugation, as now explained.

As shown in FIG. 9, the embodiment of stopper 901 has a through hole 902 that allows blood products to pass through, A, the stopper 901 when the plunger rod 904 is withdrawn for centrifugation.

As shown in FIG. 10, the embodiment of stopper 1006 has a through hole 1003 and 1004, that allows blood products to pass through, A, the stopper 1006 when the plunger rod 1001 is unscrewed from the stopper 1006 and pushed into the syringe for centrifugation while leaving the stopper 1006 in a mid-syringe or withdrawn position.

As shown in FIG. 11, the embodiment of stopper 1106 has a through hole 1103 and 1104, that allows the plunger rod 1101 to be unscrewed from the stopper 1106 and pushed into the syringe for centrifugation while leaving the stopper 1106 in a mid-syringe or withdrawn position. In addition, a through hole 1108 enables the blood products to pass through, A, the stopper 1106 when threads 1102 and 1105 are disengaged and the plunger rod 1101 is pushed into the syringe for centrifugation.

Any of the foregoing stoppers or similar configurations may, in some embodiments, be made slightly less dense than the RBC pellet and slightly more dense than the PRP. The rubber stopper may be any suitable elastomer or composite including an elastomer, wherein the density of the elastomer or composite is selected to be within the range between that of an average RBC pellet and that of an average bolus of PRP. The centrifugation process will therefore push the red blood cells below the stopper and the PRP will remain above the stopper, while the force of centrifugation will tend to push the stopper to the midpoint between the two.

After spinning the blood, the plunger rod is again configured to move the stopper, now to expel the product. In the case of the plunger rod of the type pushed through the stopper, after pulling blood from the patient into the vessel, the plunger rod is then be pulled back gently through the PRP and screwed back into the rubber stopper. Pushing the plunger rod forward then expels the PRP into a treatment site. In the case of the plunger rod of the type pulled out from the stopper and vessel, the plunger rod is reattached by gently inserting it and screwing it into the rubber stopper. The plunger rod of this embodiment is then pushed forward to expel the PRP into a treatment site. In both cases, screwing the plunger rod back into the rubber stopper reseals the hole or holes exposed by unscrewing, so that the stopper acts as a piston to move the bolus of PRP out of the vessel as desired.

### Ports and Caps

Details of the construction of the ports and other components will now be described in connection with the embodiment illustrated in FIGS. 1-3.

The ports may be implemented using Luer connections. Luer connections include Luer lock and Luer slip connections, as defined in one or more of the standards ISO 594, DIN and EN 1707:1996 and 20594-1:1993, as published at the time of this filing. Both are common and familiar on medical equipment that carries or prevents a flow of fluid. That renders them particularly suitable for application in the invention because their correct operation will be intuitive to the personnel handling the device of the invention. Both types of connection are taper connections that seal by friction locking a 6% male taper to a 6% female taper. Luer lock connections use a thread and flange arrangement to help pull the tapers together, while Luer slip connections lock directly as a result of the friction between the tapers. Other connections with suitable characteristics of asymmetry (to prevent connection errors), ease of use (to improve consistency of workflow), and reliability (to prevent leaks or other accidents) may be used.

The inflow port 102 should best be configured similarly to the inflow port on conventional syringes. It should be a male taper having a female thread arranged internally to a protective flange surrounding the male taper, to receive a hypodermic needle or other attachment having a corresponding female port with external flanges that engage the threaded flange, as is conventional. The plunger port 103 on a syringe having a male inflow port should therefore be a female port with external flanges, so as to avoid confusion with the inflow port. The caps 301, 302, for the inflow port and plunger port should therefore respectively be equipped with a female port having external flanges (inflow port cap) and a male port having a female thread internal to a flange surrounding the port (plunger port cap). Specific plunger port caps suitable for particular plunger configurations are discussed below.

If the plunger rod is of the type that is withdrawn entirely from the syringe, the cap may simply cover the plunger port as shown in FIG. 3. If the plunger rod is of the type that is detached from the stopper and pressed into the syringe, then the end of the plunger must fit within the inside diameter of the cap, which is deep enough to accommodate a length of the plunger rod that remains outside of the port when fully pressed into the syringe.

### Needles and Applicators

The system can include one or more conventional hypodermic needles (FIG. 5, 501) each of suitable gauge for either extraction of whole blood from a patient, injection of blood products or other fluids into a treatment site of a patient, or both. Such needles may have any suitable configuration for specific treatment procedures, and should have a connector as described above for connection to the inflow port of the vessel.

Alternatives to needles are also possible for specialized treatments. For example, in using the system for application of blood products or other fluids to exposed surfaces, for example the outer skin or a surgically exposed tissue, a sponge applicator may be suitable. Various shapes and configurations of sponge tips on tubular applicators may be used for a variety of procedures or treatment sites.

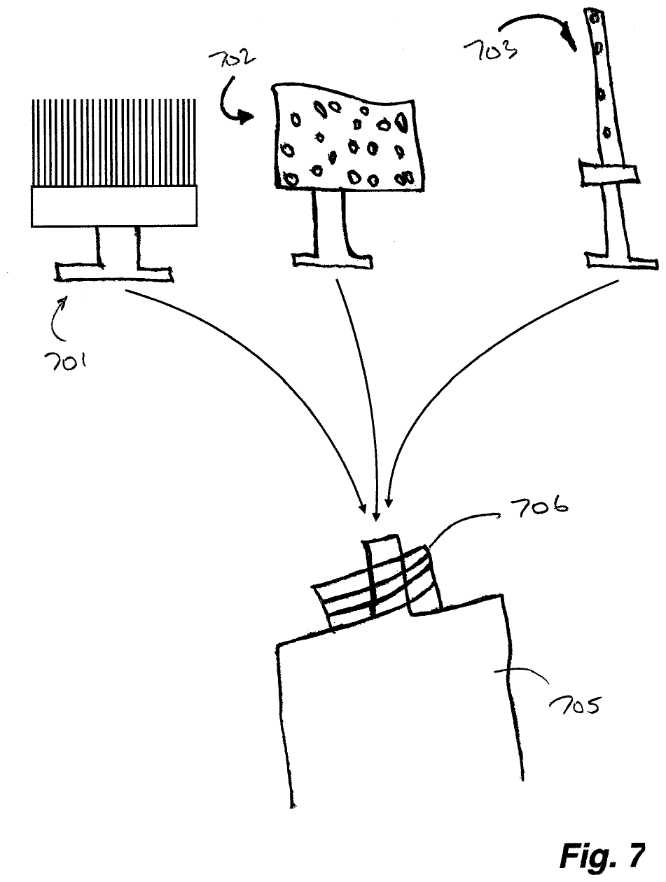

FIG. 7 shows a brush applicator 701, a sponge applicator 702, and a soft-tipped wand applicator 703, each having suitably located weep holes through which syringe contents are dispensed. Each applicator 701, 702, 703 includes a standard female Luer lock with flanges 704, for attachment to the syringe body 705 via male Luer lock 706.

### Syringe Body

As shown in FIGS. 1, 3, 4, 5, 6, and 8, the syringe 101 may include grip flanges 110 either formed as a unit with the syringe 101, or affixed by any suitable means in a fixed position. It is preferred that the grip flanges 110 be located at a distal end of the syringe 101, i.e., adjacent the inflow port 102. While the grip flanges 110 could be located adjacent the inflow port 102, adjacent the plunger port 103, or at some intermediate point along the body of syringe 101, placement adjacent the inflow port 102 is advantageous in assisting a user with properly positioning the syringe in a centrifuge with the inflow port 102 positioned so as to be the location where lighter blood products will accumulate during centrifugation.

The grip flanges of the syringe may alternatively be attached to the syringe body 801 via a sliding sleeve 802 that permits the flanges 803 to be positioned by a user at either extreme end of the syringe body, or at a central location. See, FIG. 8. This embodiment is advantageous in allowing a user to configure the syringe 801 to the user's own preference concerning the use of the grip flanges 803. When the syringe 801 is ready for centrifugation, the grip flanges 803 may be positioned adjacent the inflow port 102 as described above, so as to assist with properly positioning the syringe in a centrifuge.

### High-Efficiency Variants

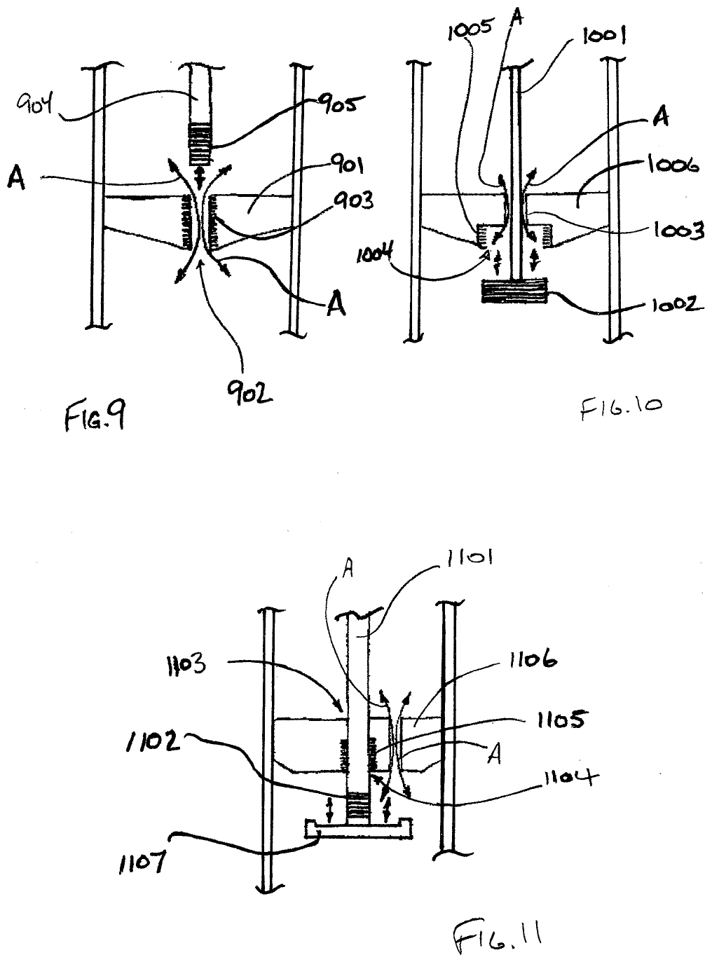

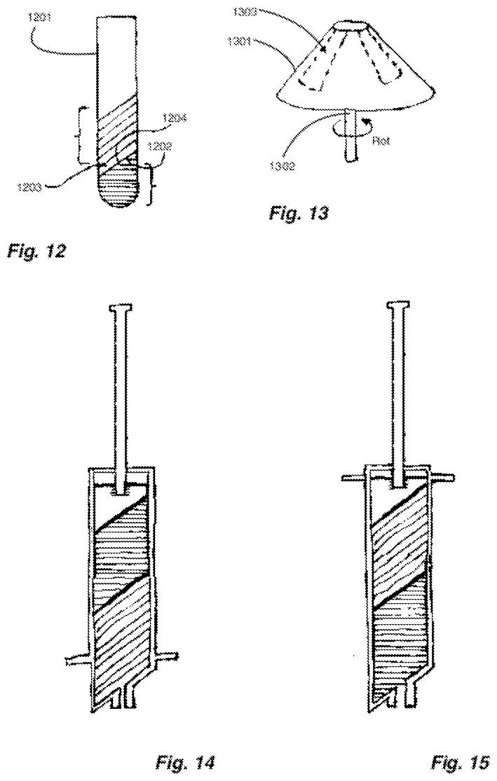

FIGS. 12 and 13 illustrate a problem with conventional medical centrifuge equipment that is also desirable to resolve in the present invention. FIGS. 14 and 15 illustrate a high efficiency syringe variant of some foregoing aspects of the invention that solve the illustrated problem. The high efficiency features now described can be combined with several of the aspects of the invention described above, as will be understood upon reading this description.

In FIG. 12, a conventional blood sample tube 1201 containing a centrifuged blood sample includes a heavy plug of RBCs 1202 below PRP 1203 that has been separated out from the sample. Note that the meniscus (line of interface) 1204 between the RBCs 1202 and the PRP 1203 is at an angle, rather than horizontal, when the sample tube 1201 is held vertically. This is a result of the construction of conventional medical centrifuges. A conventional medical centrifuge rotor 1301 is configured as shown in FIG. 13. Rotor 1301 rotates Rot about an axle 1302. Slots 1303 receive sample tubes (FIG. 12, 1201). Slots 1303 are disposed at an angle to axle 1302 that causes separated samples to exhibit an angle at the meniscus (FIG. 12, 1204) between separated components of the sample.

When PRP is separated from a patient's blood sample using the present invention, the effect illustrated in FIG. 12 causes a portion of the PRP administered back to the patient to be adulterated with RBCs from the plug of RBCs. When a syringe of the invention described above is oriented vertically (See, for example, FIG. 1), the distal, interior face of the syringe in which inflow port 102 is located and the face of elastomeric body 106 are both horizontal, not matching the angle formed by the meniscus between the RBCs and PRP when a blood sample is centrifuged (See, FIG. 12, 1204). When the meniscus is reached, some mixing between PRP and RBCs is inevitable. In at least some applications, such mixing is undesirable.

According to the variations shown in FIGS. 14 and 15, both the wall 1400a of the syringe 1400 in which a blood sample is collected and surface 1406a of stopper 1406 are angled to match the angle at which interface 1404 will form between separated PRP 1402 and RBCs 1403. The syringe 1400 is centrifuged in a position determined by which end of the syringe 1400 includes flanges 1410. Flanges 1410 may further be staggered either slightly along the length of syringe 1400, or around a circumference thereof, in order to align the syringe properly in slot 1303 of rotor 1301 shown in FIG. 13, such that the meniscus 1404 forms parallel to both stopper surface 1406a and syringe wall 1400a.

It is preferable that stopper 1406 is prevented from rotating within the syringe by either some form of keying (e.g., matched irregular shapes) or interlocking regular shapes that prevent stopper surface 1406a and syringe wall 1400a from losing their parallel orientation. Keying may be a slot longitudinally formed in the stopper that matches a ridge formed in the interior of the syringe, or any other, similarly orientation-preserving geometry. Interlocking regular shapes that prevent rotation may include stopper and syringe interior cross-sections including, but not limited to a triangle, a rectangle, a square, an oval or ellipse, or any other shape that prevents rotation.

### Kits

According to some variations, the invention may be a kit including one or more of: a plunger holder for sterile resting of the plunger rod during spinning—for embodiments having a detachable plunger; end caps that cover the ports; end caps attachable to the ports that cover the plunger and/or applicator, e.g., needle (See, FIG. 5); and a brush, sponge, or soft tip applicator for skin painting with PRP (See, FIG. 7).

### Methods and Usage

According to an exemplary method according to the invention, typical acts or steps using one of the device embodiments described above may include: inserting a hypodermic needle, butterfly, or similar into a suitable vein; attaching the inlet port via a conventional Luer lock or similar to the hypodermic needle, butterfly, or an IV line attached to either; pulling out the plunger rod so as to create a pressure gradient causing blood to flow from the vein into the syringe; either unscrewing the plunger rod from shuttlecock or stopper and removing it when syringe is full, or unscrewing the plunger rod from the shuttlecock or stopper and advancing it into the syringe without moving the stopper when the syringe is full; capping both ends of the syringe, creating a closed, sterile system to be used throughout the procedure; centrifuging the blood in a conventional, table-top or lab centrifuge while it remains in the syringe; removing the end cap from plunger rod end; returning the removed plunger rod to the syringe and screwing it back into the shuttlecock or stopper, or pulling the plunger rod previously advanced into the syringe back through supernatant and screwing it back into shuttlecock or stopper; injecting into the patient PRP from the syringe via a fresh injection needle which has been attached. Packed RBCs may be separated from the PRP by the rubber stopper in device embodiments having a stopper with a hole, or packed RBCs may simply be pressed forward by the rubber stopper without creating an admixture with the PRP. Density controlled gel can alternatively be placed in the syringe prior to centrifugation to separate platelets from PRP for clinical applications in which this is desired.

According to a one particular embodiment of the inventive method, for taking a biopsy and simultaneously treating the suspect tissue and needle track areas, the foregoing steps can be practiced as follows. The syringe can be prepared by first performing the steps of extracting blood from the patient, performing centrifugation, and reattaching the plunger to the shuttlecock or stopper. The fresh injection needle may be a coaxial needle set that combines an inner core biopsy needle with an outer cannula through which an injection may be made after the core biopsy needle is withdrawn. The coaxial needle set is inserted through the skin and guided using radiologic or sonographic imaging guidance methods and tools into the suspect tissue area; the inner core biopsy needle is then withdrawn with the tissue sample; the syringe is then attached via the Luer lock or similar connection to the outer cannula, while the outer cannula remains in place; and then the outer cannula is withdrawn from the patient while simultaneously injecting PRP into the needle track. This method deposits a therapeutically significant track of PRP in the suspect tissue and the needle track, thus aiding in the clotting, sealing, and healing of the needle puncture.

According to another embodiment of the method, for taking a biopsy and simultaneously treating the suspect tissue and needle track areas, the additional steps now described may be practiced. The patient blood extracted into the syringe may be treated with additional therapeutically active agents that mix with the PRP and are then deposited into the needle site along with the PRP during injection of the PRP as described above.

According to further embodiments of the method, the red blood cells can be removed from the back of the syringe according to the invention using a second syringe. The volume formerly occupied by the removed red blood cells can be replaced by any one or more of a variety of therapeutically active agents, including chemotherapy agents, stem cells, exosomes of various sorts, other chemical messengers, anti-cancer viruses and antibodies, antibiotics, anti-fungals, brachytherapy agents, and combinations of the foregoing. Modifiers such as gel carriers to help prevent migration of therapeutics and fiducial markers could be included as well. By using such agents in a syringe and method as described, therapeutic treatment of a presumptive diagnosis may begin immediately at the time a tissue biopsy is taken by needle.

### Clinical Practice

In very brief summary, using the syringe as described above, a procedure as follows can be performed with no transfers of materials between sterile vessels. A single syringe: collects a specimen; holds the specimen while it is centrifuged; holds the specimen while removing a top 1 cc of saline, e.g., by connecting the plunger port to another syringe and pushing up from bottom with the syringe upright; receives therapeutic agents such as stem cells, immunologic agents, or antibiotics into the PRP; and, finally injects the treated PRP roughly up to the point of the RBCs. The entire contents of the syringe could be injected if desired, but the RBC is superfluous to most treatments. The therapeutically active portion of most treatments will be within the first 6 cc of treated PRP. In some exemplary therapies that will be described, the PRP forms a fibrin scaffold which holds and delivers an adjuvant therapeutically active treatment agent. The adjuvant therapeutically active treatment agent may be a biologic or non-biologic agent, depending on the condition to be treated and the treatment strategy and plan. The combination of PRP and an adjuvant is referred to hereinafter as PRP+.

Biologics are agents made from living organisms or the products of living organisms. Biologics may include, but are not limited to, stem cells, vaccines, antibodies, etc. Biologics may include sources of exosomes, various known and unknown tissues, and other products such as amniotic fluid.

Non-biologics are agents made from non-living source material not the product of a living organism. Non-biologics may include, but are not limited to, radioactive agents for brachytherapy, high-density markers for external beam radiotherapy, immunologic response stimulators, tumor antigens, antiviral and antibiotic agents, anti-inflammatory agents, etc.

Biologics and non-biologics may be provided as liquids or fine particulates, nano-particles, DNA, RNA, anti-mutatgenic agents, anti-metastatic agents, and time-released agents.

Treatments may alternatively be based on platelet poor plasma, white blood cells, or fibrin gel as a treatment or medium to carry a treatment agent instead of PRP. PRP and PRP+ are, in this application, therefore exemplary and not exclusive.

### Cellular Signaling and Genetic Coding Agents

DNA and RNA are both cellular signal agents as well as genetic coding agents for genetic information transfer, i.e., cell programming, as well as process modulating agents. They play a role regulating cellular metabolism as well. Cells exchange DNA, each to help another regulate behavior. Using DNA editing technology, such as CRISPER, any DNA desired to cause a cell to behave in a particular way may be inserted. Such technology can tell cancer cells to stop being cancer cells. Such technology can tell cancers not to metastasize. Such technology give cells a gene drive to tell the cell line to die out. Such technology can tell the cancer cells to drop their immune down regulation. Such technology can tell them to present antigens that human immune systems can recognize and kill. Such technology can create tutor cells to teach cancer stem cells to stop making more stem cells. Thus, using the syringe taught above to deliver treated cells as an adjuvant in PRP+ can place known cancer treatments such as described above in a stable bolus directly adjacent or within the tissue to be treated.

Alternatively, the immune system can similarly be manipulated to more readily recognize and destroy a cancer. Treated cells containing the DNA or RNA instructions for identifying and destroying cancerous tissue can be created and added to PRP as an adjutant, creating a PRP+ to condition a human immune system to recognize and destroy cancer.

### Autoimmune Diseases

DNA, RNA, and protein signals can be incorporated in PRP+ as an adjutant to down regulate the human immune response. Thus, for example, if a lung nodule is autoimmune, such as a rheumatoid nodule, PRP+ can be injected directly into the nodule to tell the nodule to stop up regulating an overactive immune response that is responding improperly to the body in which the nodule exists.

Similarly, bits of cells, exosomes, or other nano-vesicles can be incorporated as an adjuvant of PRP+ that tell an immune system to stop allergies or to turn off asthma.

### Healing

Adjuvants are known that promote healing. These can also be administered in a targeted manner using the syringe disclosed above, with remarkable healing effects.

A healing adjuvant can instruct lung cells damaged by COPD to stop destroying lung tissue. Such an adjuvant, injected in a desired location within a damaged lung can further up-regulate lung cells to make new lung tissue.

The needle of the syringe disclosed above may be inserted into a wound, a bone, or a scar and inject PRP+ having an adjuvant telling it to regenerate tissues.

Similarly, inserting the needle into the brain, spinal cord, or other nerve can with suitably selected DNA, RNA, or signaling proteins and cellular machinery, tell nerve cells to bridge a gap in the circuit. Reconnecting nerves in trauma cases, paralysis cases caused by nerve or spinal damage, and stroke can dramatically benefit patients through targeting the new therapeutic agents now available directly into the injured tissue.

Signaling DNA, RNA, or signaling proteins could be injected into the eyes, teaching the cells of the retina to make new eye sensing cells, so as to reverse macular degeneration.

Intervertebral discs that have degenerated with age or activity can be repaired by methods employing the syringe described. The method can place healthy stem cells in the proper location to encourage growth of normal discs.

### Alzheimer's Disease

People with Alzheimer's disease may benefit from local brain regrowth. Currently, Parkinson's disease is treated using deep brain stimulators. Similarly, and analogously to the treatments described above, the syringe disclosed above can deliver cellular instructions to make new brain tissue at the time of probe placement.

### Other Delivery Adjuvants

Viruses that have been suitably altered to implant useful DNA at locations that might benefit from such useful DNA can turn a patient's own cells into a factory to make useful proteins. For example, a suitably altered virus can insert DNA into cancer stem cells instructing the stem cells to be non-mutated, non-cancerous stem cells. In another example, a suitably altered virus can insert DNA into the cells of a Tay-Sachs disease patient, turning on those cells' ability to make hexosaminidase A.

Just as insulin is now manufactured using external biologic processes, a patient's own pancreas can be instructed by a biologic adjuvant to make the patient's own insulin.

### Clinical Summary

Any disease tissue or injured tissue that is organ-specific or otherwise localizable can have a fibrin scaffold inserted and filled with packets of code and supporting cells and signals to cause the disease process to change or cease, or to cause healing.

Stem cells need a three-dimensional matrix on which to grow. They also need an extra-cellular matrix made by fibroblasts to thrive. It is now known to make tiny packets that include the nurse cells, the matrix, the signals and the code to fix a biological problem, and the above-described syringe can safely deliver those packets directly to the tissue that it is desired to affect.

### Other Cell Behavior Alterations

According to some methods, the syringe described above may be used to inject agents that help render cells photosensitive so they can be destroyed for example by laser light. The combination of adjuvants injected in PRP+ into or in the vicinity of a tumor may, for example, include a laser-absorptive dye and cell signaling materials as described above instructing cells to take up the dye at a higher than normal rate. The PRP+ could in addition include in the payload of adjuvants include a visualization aid, such as a UV-sensitive dye or density-altering material. Thus, after administering the PRP+ using the syringe described above, a physician or other operator could use a UV lamp or other device depending on the visualization aid employed to visualize all the metastases of the tumor. In the case of the example payload of adjuvants, a laser could then be used to target all the visualized cells which after a suitable time have taken up the laser-absorptive dye. Those cells would then be selectively destroyed without causing significant damage to adjoining tissues. That is, the administration of the PRP+ to the main tumor teaches the cells of the main tumor to take up the dye and the cells of the main tumor then teach all the metastases to also take up the dye.

A different PRP+ adjuvant payload could teach the cells of a human host to secrete a taste or a smell protein, so when the cell is altered to the disease state, and the disease thus returns, the secretion is turned on and the person can self-sense the return of the disease.

Adjuvants to PRP+ available today could turn off a local pain pathway.

In utero surgery can be revolutionized by enabling a surgeon to inject PRP+ into an abnormally developing heart, limb, etc., so as to deliver instructions to repair the developmental defect. Such surgery could then be as straight-forward as a sonogram-guided injection into the affected part.

What is claimed is:

1. A syringe, comprising:
a syringe vessel having a port at a distal end and a port at a proximal end;
a movable stopper disposed within the syringe vessel;
a detachable plunger rod fitting through the port at the proximal end; and
an interconnection structure that permits attaching the detachable plunger rod to the movable stopper without applying pressure sufficient to move the stopper.

2. The syringe of claim 1, wherein:
the syringe vessel has a male Luer lock at the port at the distal end and a female Luer lock at the port at the proximal end;
the movable stopper includes a threaded shuttlecock disposed within the syringe vessel;
the detachable plunger rod fits through the female Luer lock and has threads to engage the threaded shuttlecock; and further comprising administration flanges at the distal end adjacent the male Luer lock.

3. The syringe of claim 1, further comprising:
a hole defined in the movable stopper, the hole having a locking contour; and
a corresponding locking contour defined on an end of the plunger rod.

4. The syringe of claim 3, wherein:
the locking contour is selected from one of a thread and a bayonet mount.

5. The syringe of claim 3, wherein the moveable stopper has defined therethrough an exposable hole through which blood product may pass.

6. The syringe of claim 5, for separating a blood component having a first density from a blood product having a second density by centrifugation, wherein the stopper has a third density between the first and second densities, and the stopper slides freely within the syringe vessel during centrifugation.

7. The syringe of claim 5, wherein the through hole is the exposable hole.

8. The syringe of claim 7, wherein the locking contour and the corresponding locking contour disengage so as to permit the detachable plunger rod to be pushed through the stopper.

9. The syringe of claim 8, further comprising:
a cap for the port at the proximal end capable of covering the plunger rod when disengaged from the stopper.

10. The syringe of claim 3, further comprising:
a cap for the port at the proximal end when the plunger rod is disengaged from the stopper; and
a cap for the port at the distal end; wherein
the syringe is a closed, sterile vessel suitable for centrifugation and other processing with the caps applied.

11. The syringe of claim 10, the port at the proximal end further comprising:
the port constructed and arranged to accept infusion apparatus for infusing agents into the closed, sterile vessel.

12. The syringe of claim 1, wherein the distal end of the syringe is defined by a wall having an angle matching an angle formed by a meniscus between blood fractions after centrifugation, and the stopper has a surface at an angle also matching the angle formed by the meniscus.

13. The syringe of claim 12, wherein the stopper is keyed to the syringe so as to maintain an orientation between the surface of the stopper at the angle matching the meniscus and the wall having the angle matching the angle formed by the meniscus without rotation.

14. A method of treating a patient using the syringe of claim 2, comprising:
drawing blood from the patient into the syringe while a first needle is affixed to the male Luer lock and the plunger rod is threaded to the shuttlecock;
centrifuging blood in the syringe after removing the first needle and the plunger rod and capping both the male Luer lock and the female Luer lock; and
re-administering a portion of the partitioned blood directly from the syringe after attaching a second needle to the male Luer lock and re-threading the plunger rod to the shuttlecock.

15. A method of treating a patient with a derivative blood product using the syringe of claim 1, comprising:
drawing blood from the patient into the syringe vessel while a first needle is affixed to the port at the distal end and the plunger rod is engaged to the movable stopper;
removing the first needle from the port at the distal end;
disengaging the plunger rod;
capping both the port at the distal end and the port at the proximal end;
centrifuging blood in the syringe after removing, disengaging, and capping, until the blood is partitioned;
attaching an applicator to the port at the distal end; and
administering a portion of the partitioned blood directly from the syringe after attaching and re-threading the plunger rod to the movable stopper.

16. The method of claim 15, for treating a patient with autologous platelet-rich plasma (PRP), further comprising:
partitioning PRP from concentrated red cells during centrifugation.

17. The method of claim 15, for treating a patient with exosomes, comprising:
partitioning blood components containing exosomes from other blood components during centrifugation.

18. The method of claim 15, for treating a patient with stem cells, comprising:
partitioning blood components containing stem cells from other blood components during centrifugation.

19. The method of claim 16, for preventing hemorrhaging along a biopsy needle path in a needle biopsy patient, wherein the applicator is a syringe needle, administering comprising:
injecting the PRP into the biopsy needle path.

20. The method of claim 16, for treating a patient for cancer, wherein the applicator is a syringe needle, administering comprising:
injecting the PRP into a cancer site.

21. The method of claim 18, for treating a patient for aging-related conditions, wherein the applicator is a syringe needle, administering comprising:
injecting the stem cells into a patient site needing renewal.

22. The method of claim 16, for treating a patient for topical skin conditions, wherein the applicator is a dispensing tip, administering comprising:
dispensing and distributing the PRP onto a patch of skin exhibiting the topical skin condition.

23. The method of claim 16, for treating a patient using an adjuvant comprising at least one of a genetic coding agent, a cellular signaling agent, a marker agent, a radiotherapy agent, an immunologic response stimulating agent, a tumor antigen agent, an antiviral agent, an antibiotic agent, and an antimutagenic agent, further comprising:
infusing the adjuvant into the PRP, producing PRP+; and
injecting a therapeutic quantity of the PRP+ into a site requiring treatment.

24. A method of treating a patient with a derivative body fluid product using the syringe of claim 1, comprising:
drawing a body fluid from the patient into the syringe vessel while a first needle is affixed to the port at the distal end and the plunger rod is engaged to the movable stopper;
removing the first needle from the port at the distal end;
disengaging the plunger rod;
capping both the port at the distal end and the port at the proximal end;
centrifuging the body fluid in the syringe after removing, disengaging, and capping, until the body fluid is partitioned;
attaching an applicator to the port at the distal end; and
administering a portion of the partitioned body fluid directly from the syringe after attaching and re-threading the plunger rod to the movable stopper.
